(19) United States
(12) Patent Application Publication
(10) Pub. No.: US 2025/0177434 A1
(43) Pub. Date: Jun. 5, 2025

(54) ANTIVIRAL AGENTS FOR TREATMENT OF CORONAVIRUSES

(71) Applicant: Meharry Medical College, Nashville, TN (US)

(72) Inventor: Donald J Alcendor, Hermitage, TN (US)

(73) Assignee: Meharry Medical College, Nashville, TN (US)

(21) Appl. No.: 18/921,511

(22) Filed: Oct. 21, 2024

Related U.S. Application Data
(63) Continuation of application No. 17/244,272, filed on Apr. 29, 2021, now Pat. No. 12,121,533.
(60) Provisional application No. 63/017,603, filed on Apr. 29, 2020.

(51) Int. Cl. A61K 31/7125; A61K 47/64; A61P 31/14

(52) U.S. Cl. CPC A61K 31/7125; A61K 47/641; A61P 31/14

(57) ABSTRACT

An antiviral agent is provided, having a phosphorodiamidate morpholino oligomer with an antisense sequence to a portion of a genome of a strain of a coronavirus. The coronavirus may be SARS-CoV-2 or another βCoV. The antiviral agent finds many uses, such as in a pharmaceutical composition, a method of treating coronavirus-mediated disease, a method of preventing coronavirus-mediated disease, a method of reducing or preventing the replication of coronavirus in a host cell, a method of controlling the spread of coronavirus in donated tissue, a treated tissue sample, and in the manufacture of a medicament for the treatment or prevention or coronavirus-mediated disease.

### CROSS-REFERENCE TO RELATED APPLICATIONS

This application is a continuation of U.S. application Ser. No. 17/244,272, filed on Apr. 29, 2021, now U.S. U.S. Pat. No. 12,121,533, which claims the benefit of U.S. Provisional Patent Application No. 63/017,603, filed Apr. 29, 2020, both disclosures of which are incorporated by reference herein in their entirety.

### INCORPORATION-BY-REFERENCE OF SEQUENCE LISTING

The material in the accompanying sequence listing is hereby incorporated by reference in its entirety into this application. The accompanying file, named 212149-401095 Sequence Listing.xml, was created on Feb. 26, 2025 and is 95 KB.

### BACKGROUND

### A. Field of the Disclosure

The present disclosure relates generally to medicine, specifically to antiviral agents, and particularly antiviral agents for the treatment of coronaviruses, such as SARS-CoV-2. Such agents, methods of use, and kits for use therefor are provided.

### B. Background

Severe acute respiratory syndrome coronavirus 2 (i.e., “SARS-CoV-2”) is a strain of virus that causes coronavirus disease 2019 (i.e., COVID-19). SARS-CoV-2 is a novel coronavirus that is highly transmissible and pathogenic for humans (and potentially other animals) and can cause life-threatening illness (particularly respiratory disease), especially in individuals with underlying comorbidities or are otherwise in a sensitive group (e.g., immunocompromised or immunosuppressed). Because of the effects of SARS-CoV-2 on an infected individual's respiratory system, a percentage of those infected require breathing support, such as by oxygen infusion and/or a mechanical ventilator. On Mar. 11, 2020, the Worldwide Health Organization (“WHO”) declared COVID-19 to be a pandemic—the first time a pandemic had been declared by WHO since H1N1 (the virus that causes the disease known as “swine flu”) in 2009. This declaration tracked the global spread of the virus, with national and local governments worldwide enacting various social distance measures to limit the spread of COVID-19 in an effort to avoid overwhelming medical capacity and resources (such as available hospital beds and ventilators).

Coronaviruses (CoVs) are positive-sense, single-stranded, enveloped, RNA viruses that belong to the subfamily Coronavirinae, family Coronavirdiae, order Nidovirales and are classified into four genera of CoVs: namely, Alphacoronavirus (αCoV), Betacoronavirus (βCoV), Deltacoronavirus (δCoV), and Gammacoronavirus (γCoV). To date, seven human coronaviruses (HCoVs) have been identified, including two α-CoVs (HCoV-229E and HCoVNL63) and five β-CoVs (HCoV-OC43, HCoV-HKU1, severe acute respiratory syndrome CoV (SARS-CoV), Middle East respiratory syndrome CoV (MERS-COV), and most recently β-CoV SARS-CoV-2 (COVID-19). WHO has classified COVID-19 as a βCoV of group 2B. CoVs cause respiratory, enteric, hepatic, and neurological diseases in humans among other different animal species, including camels, cattle, cats, and bats. The βCoV lineage HCoV-OC43 and HCoV-HKU1 are typically associated with self-limiting upper respiratory infections in immunocompetent hosts and occasionally lower respiratory tract infections in immunocompromised hosts and the elderly. Coronaviruses possess the largest genomes of all RNA viruses, consisting of about 30 kb.

SARS-CoV-2 belongs to the βCoV genera and has 89% nucleotide identity with bat SARS-like-CoVZXC21 and 82% with that of human SARS-CoV. Examinations of the viral evolution show that bats and rodent are gene sources for most αCovs and βCoVs while avian species are the proposed gene sources of most δCoVs and γCoVs. CoVs often cross species barriers to infect humans and have emerged to cause significant morbidity and mortality in the general population. The most recent examples are severe acute respiratory syndrome CoV (SARS-CoV) that emerged in China in 2002 with 8000 infections and 800 deaths (generally known as the SARS epidemic) and the and Middle East Respiratory Syndrome CoV (MERS-COV) that emerged in the Arabian Peninsula since 2012. Developing an antiviral for COVID-19 requires consideration of its genetic complexities as well as the potential to mutate in humans or a zoonotic intermediate host.

Currently, there are no FDA approved treatments for COVID-19, particularly treatments that are effective against SARS-CoV-2. Moreover, there is no current vaccine to prevent COVID-19. This represents an urgent unmet medical need for efficacious therapeutics for COVID-19. Even if a vaccine were to be developed, there is an acute and short to medium-term need in the meantime for therapeutic interventions to treat COVID-19 and/or prevent or reduce transmission. Moreover, a treatment effective against SARS-CoV-2 is needed for other animal hosts, whether to eliminate zoonotic host-mediated infection or to reduce to the risk of mutation in a zoonotic intermediate host to a novel strain of coronavirus.

### SUMMARY

The problems expounded above, as well as others, are addressed by the invention of an antiviral agent that effectively prevents the replication of strains of coronaviruses, including SARS-CoV-2 (although it is to be understood that not all such problems will be addressed by every such embodiment).

In a first aspect, an antiviral agent is provided, the antiviral agent comprising a phosphorodiamidate morpholino oligomer comprising an antisense sequence to a portion of a genome of a strain of a coronavirus (such as a strain of SARS-CoV-2).

In a second aspect, a pharmaceutical composition for the treatment or prevention of a disease mediated by a coronavirus (such as SARS-CoV-2) is provided, the composition comprising: the antiviral agent above and a pharmaceutically acceptable carrier.

In a third aspect, a method of treatment or prevention of a disease mediated by a strain of a coronavirus (such as SARS-CoV-2) in a subject in need thereof is provided, the method comprising administering to the subject a therapeutically effective amount of the antiviral agent or the pharmaceutical composition above.

In a fourth aspect, a method of reducing or preventing the replication of a strain of a coronavirus (such as SARS-CoV-2) in a host cell is provided, the method comprising contacting the host cell with the antiviral agent above.

In a fifth aspect, a method of controlling the spread of a strain of a coronavirus (such as SARS-CoV-2) in donated tissue is provided, the method comprising exposing the donated tissue to an effective amount of the agent above.

In a sixth aspect, a treated donated tissue sample is provided, comprising a sample of donated tissue and the agent above.

In a seventh aspect, a use of the agent above in the manufacture of a medicament for the treatment or prevention of a disease mediated by a strain of a coronavirus (such as SARS-CoV-2) is provided.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

### BRIEF DESCRIPTION OF THE DRAWINGS

FIG. 1: Sequences of the 5′UTR for SARS-CoV-2, the bat SARS-like-CoVZXC21, and SARS-CoV are depicted, with the start site for pp1a (atg) shown in highlighting with white text.

FIG. 2A: A genome organization of βCoV is illustrated.

FIG. 2B: A schematic structure of a vivo-morpholino composed of a 25-mer long morpholino oligonucleotide covalently linked to an octaguinidine dendrimer, which serves as a delivery moiety. A nucleotide sequence of SARS-CoV-2 (2019-nCoV_KHU-SZ-005b_2020 vivo-morpholino (i.e., MRCV-19) that is complementary to SARS-CoV-2 target sequence is also shown.

### DETAILED DESCRIPTION

### A. Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well-known functions or constructions may not be described in detail for brevity or clarity.

The terminology used herein is for the purpose of describing particular embodiments only and is not intended to be limiting. As used herein, the singular forms “a,” “an,” and “the” are intended to include the plural forms as well, unless the context clearly indicates otherwise.

The terms “first,” “second,” and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

The term “consisting essentially of” means that, in addition to the recited elements, what is claimed may also contain other elements (steps, structures, ingredients, components, etc.) that do not adversely affect the operability of what is claimed for its intended purpose as stated in this disclosure. This term excludes such other elements that adversely affect the operability of what is claimed for its intended purpose as stated in this disclosure, even if such other elements might enhance the operability of what is claimed for some other purpose.

The terms “about” and “approximately” shall generally mean an acceptable degree of error or variation for the quantity measured given the nature or precision of the measurements. Typically, exemplary degrees of error or variation are within 20 percent (%), preferably within 10%, and more preferably within 5% of a given value or range of values. For biological systems, the term “about” refers to an acceptable standard deviation of error, preferably not more than 2-fold of a given value. Numerical quantities given herein are approximate unless stated otherwise, meaning that the term “about” or “approximately” can be inferred when not expressly stated.

The terms “prevention,” “prevent,” “preventing,” “suppression,” “suppress,” and “suppressing,” as used herein, refer to a course of action (such as administering a pharmaceutical composition) initiated prior to the onset of a clinical manifestation of a disease state or condition so as to reduce its likelihood or severity. Such a reduction in likelihood or severity need not be absolute to be useful.

The terms “treatment,” “treat,” and “treating,” as used herein, refer to a course of action (such as administering a pharmaceutical composition) initiated after the onset of a clinical manifestation of a disease state or condition so as to eliminate or reduce such clinical manifestation of the disease state or condition. Such treating need not be absolute to be useful.

The term “in need of treatment,” as used herein, refers to a judgment made by a caregiver that a patient requires or will benefit from treatment. This judgment is made based on a variety of factors that are in the realm of a caregiver's expertise, but that judgment includes the knowledge that the patient is ill, or will be ill, as the result of a condition that is treatable by a method or device of the present disclosure.

The term “in need of prevention,” as used herein, refers to a judgment made by a caregiver that a patient requires or will benefit from prevention. This judgment is made based on a variety of factors that are in the realm of a caregiver's expertise, but that judgment includes the knowledge that the patient will be ill or may become ill, as the result of a condition that is preventable by a method or device of the disclosure.

The terms “individual,” “subject,” or “patient,” as used herein, refer to any animal, including mammals, such as mice, rats, other rodents, rabbits, dogs, cats, swine, cattle, sheep, horses, primates, and humans. The term may specify male or female or both, or exclude male or female.

The term “therapeutically effective amount” (or simply “effective amount”), as used herein, refers to an amount of an agent, either alone or as a part of a pharmaceutical composition, that is capable of having any detectable, positive effect on any symptom, aspect, or characteristics of a disease state or condition. Such an effect need not be absolute to be beneficial.

The term “pharmaceutically acceptable salts,” as used herein, includes salts of the antiviral agents which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogen carbonic, phosphoric, monohydrogen phosphoric, dihydrogen phosphoric, sulfuric, monohydrogen sulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts,” Journal of Pharmaceutical Science, 1977, 66, 1-19). Certain specific compounds of the present invention contain both basic and acidic functionalities that allow the compounds to be converted into either base or acid addition salts.

Nucleic acids are “complementary” to each other, as used herein, when a nucleotide sequence in one strand of a nucleic acid, due to orientation of its nucleotide hydrogen atoms, hydrogen bonds to another sequence on an opposing nucleic acid strand (of course, a strand of a nucleic acid may be self-complementary as well). The complementary bases typically are, in DNA, A with T, and C with G, and, in RNA, C with G, and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementarity means that a sequence in one strand is not perfectly complementary to a sequence in an opposing strand, but that sufficient bonding occurs between bases on the two strands to form a stable hybrid complex at a given set of hybridization conditions (e.g., salt concentration and temperature). Such conditions can be predicted by using the sequences and standard models to predict the Tₘ of hybridized strands, or by empirical determination of Tₘ by using established methods. Tₘ refers to the temperature at which a population of hybridization complexes formed between two nucleic acid strands are 50% denatured. At a temperature below the Tₘ, the formation of a hybridization complex is favored, whereas at a temperature above the Tₘ, melting or separation of the strands in the hybridization complex is favored. Such stringency is based on the melting temperature (Tₘ) of the nucleic acid binding complex, as taught in Berger and Kimmel (1987, Guide to Molecular Cloning Techniques, Methods in Enzymology, 152, Academic Press, San Diego CA). The Tₘ of an annealed duplex depends on the base composition of the duplex, the frequency of base mismatches, and the ionic strength of the reaction medium. The Tₘ of a duplex can be calculated by those of ordinary skill in the art based on these two factors using accepted algorithms. Maximum stringency typically occurs at about 5° C. below Tₘ; high stringency at about 5-10° C. below Tₘ; intermediate stringency at about 10-20° C. below Tₘ; and low stringency at about 20-25° C. below Tₘ. As will be understood by those of skill in the art, a maximum stringency hybridization can be used to identify or detect identical nucleotide sequences while an intermediate (or low) stringency hybridization can be used to identify or detect similar or related sequences. Terms such as maximally stringent, highly stringent, and poorly stringent, refer to conditions of maximal stringency, high stringency, and low stringency respectively.

In the following discussion certain outside documents are referenced to enable the reader to make and use the subject matter described herein. Nothing contained herein is to be construed as an “admission” of prior art. Applicant expressly reserves the right to demonstrate, where appropriate, that such documents referenced herein do not constitute prior art under the applicable statutory provisions.

### B. Antiviral Agents

A phosphorodiamidate morpholino oligomer (PMO) is disclosed that suppresses viral replication in strains of coronaviruses, such as SARS-CoV-2. In the interest of clarity, not all features of an actual implementation are described in this specification. It will, of course, be appreciated that in the development of any such actual embodiment, numerous implementation-specific decisions must be made to achieve the worker's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

PMOs are nucleic acids having conventional nucleotide bases, but a backbone of methylenemorpholine rings and phosphorodiamidate linkages. PMO bind to RNA with high specificity. This gives PMOs the ability to block the translation of mRNA by binding to complementary sequences on the mRNA, which prevents binding of the mRNA to the ribosome. Translational blocking with PMOs is highly specific and does not result in blocking of non-target mRNA. PMOs are also much more stable than RNA and resistant to most exonucleases. An unmodified PMO has the following general structure, with each “B” being independently selected from adenine, cytosine, guanine, or thymine:

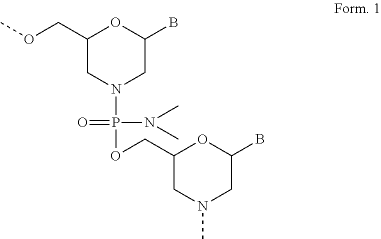

The PMO of the antiviral agent comprises a nucleotide sequence that is complementary to a sequence in a viral genome (the “target sequence”). Such a complementary sequence is referred to herein as the “antisense sequence,” although as explained below, in some embodiments the sequence may deviate from an exact antisense sequence of the target. The genome may be, without limitation, the genome of a single-stranded positive sense RNA virus, such as a flavivirus or coronavirus. In a specific embodiment of the agent, the genome is a genome of a strain of a coronavirus, such as SARS-CoV-2. The sequence in the viral genome should be a sequence that must bind to the cellular ribosome for replication to occur. This may be a sequence in a structural gene (i.e., in an open reading frame), or it may be a non-translated sequence that facilitates the binding of the strand to the ribosome.

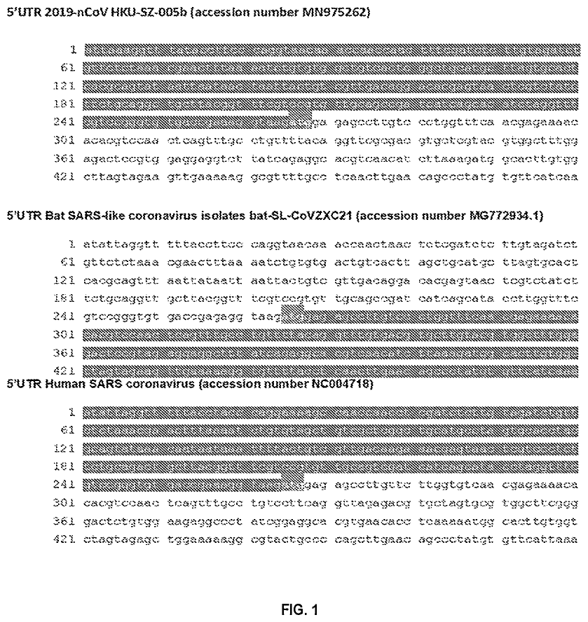

For purposes of illustration, the SARS-CoV-2 genome will be used as an example. The SARS-CoV-2 genome comprises an untranslated 5′ region (“UTR”) translation initiation complex that overlaps with the pp1a start site of translation. It is believed that this portion of the SARS-CoV-2 genome is higher conserved (i.e., shares a high degree of identity) among βCoV, including SARS-CoV, SARS-CoV-2, and bat SARS-like-CoVZXC21 (FIG. 1). The SARS-CoV-2 genome has been assigned GENBANK accession number MN975262, and is incorporated herein by reference in its entirety (SEQ ID NO: 5). The SARS-CoV genome has been assigned GENBANK accession number AY278741, and is incorporated herein by reference in its entirety (SEQ ID NO: 6). The bat SARS-like-CoVZXC21 genome has been assigned GENBANK accession number MG772934, and is incorporated herein by reference in its entirety (SEQ ID NO: 7). The ZIKV genome is flanked by a 5′ untranslated region (UTR) and 3′ UTR. Without wishing to be bound by any hypothetical model, the interaction between 5′ and 3′ UTRs are believed to be critical for viral RNA replication, with sequences across the UTRs being highly conserved across strains of coronaviruses and specifically βCoV.

In some embodiments of the agent, the target sequence is a sequence from the 5′ region of a strain of coronavirus (such as SARS-CoV-2) genome, for example, the region encompassing the pp1a gene region and the 5′ untranslated region (UTR). In a specific embodiment of the agent, the target sequence comprises 5′-AGG TAA GAT GGA GAG CCT TGT CCC T-3′ (SEQ ID NO: 2) from the 5′ UTR. In the same specific embodiment, the PMO comprises the sequence 5′-AGG GAC AAG GCT CTC CAT CTT ACC T-3′ (SEQ ID NO: 1).

In a specific embodiment of the agent, the PMO comprises the sequence 5′-AAG AAC AAG GCT CTC CAT CTT ACC T-3′ (SEQ ID NO: 3) the targets 5′-GCT GGG AAA GAC CAG AGA CTC CAT G-3′ (SEQ ID NO: 4). In some embodiments, the antisense sequence will bind with high stringency to the target sequence under physiological (intracellular) conditions. Such conditions are understood by those of ordinary skill in the art but will vary by cell type. For example, intracellular pH and sodium concentration vary in a narrow range by cell type. Physiological conditions for human subjects are generally at 37° C. (98.6° F.). Typically, this means that the antisense sequence will have at least 80% identity with an exact complement of the target sequence. In various embodiments of the agent the antisense sequence will have at least 70%, 85%, 90%, 95%, 96%, 97%, 98%, or 99% identity with an exact complement of the target sequence. In a specific embodiment the antisense sequence is an exact complement of the target sequence.

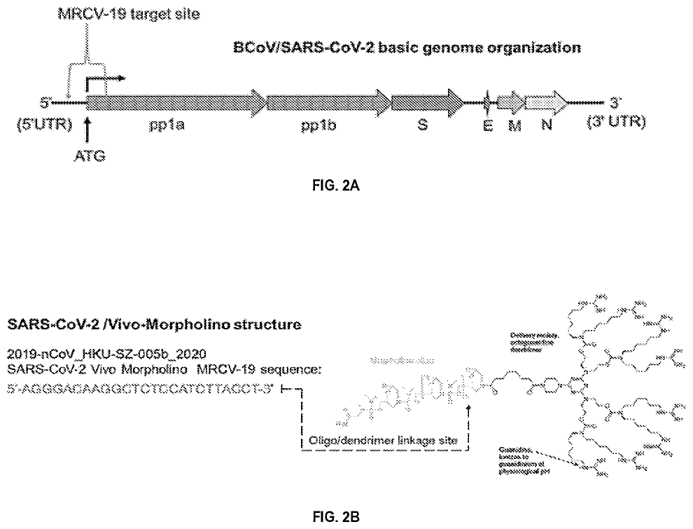

The antisense sequence will generally be about 25 bases long or exactly 25 bases long. In some embodiments, the length of the antisense sequence can vary somewhat, in the range of about 10-30 bases. Specific embodiments of the antisense sequence can be any length from 10-30 bases. More specific embodiments are 15-25 bases. A particular embodiment of antisense sequence is exactly 25 bases long. The PMO may comprise additional nucleotides on the 5′ end or 3′ end (or both) of the target recognition sequence. In a specific embodiment, the antisense sequence is the entire nucleotide sequence of the PMO, and there are no additional nucleotides on the 5′ end or the 3′ end of the antisense sequence. Advantageously, it has been discovered that portions of 5′ UTR sequences among different coronaviruses, and in particular βCoV, are highly conserved and share a high degree of identity. Referring to FIGS. 1-2B, the sequences representing the 5′UTR for SARS-CoV-2, the bat SARS-like-CoVZXC21, and SARS-CoV are shown (FIG. 1). The start site for pp1a (atg) is shown in highlighting with white text (FIG. 1). A complementary sequence that overlaps the 5′UTR and the regions immediately downstream of the ATG start site was identified as the target to engage the SARS-CoV-2 virus upon entry into permissive cells in vitro and in vivo based on its genome structure (FIG. 2A). The structure of the in vivo morpholino and the complementary sequence targeting SARS-CoV-2 genome are shown in FIG. 2B.

The PMO may have other various desirable characteristics. These may include without limitation: a base sequence that has very little self-complementarity; a high enough GC-content (guanine-cytosine content) (e.g. 40-60%) so that it has a high target affinity; and no stretches of four or more contiguous G to preserve water solubility.

The PMO may have modified 3′ or 5′ ends to add various additional functionalities. Such modifications can include 3′ conjugation with any of: a fluorophore, a quencher, carboxyfluorescein, lissamine, dabcyl, biotin, amine, amine with biotin, disulfide amine, pyridyl dithio, azide, and alkyne. Such modifications may include 5′ conjugation with any of: a primary amine, dabcyl, azide, and alkyne. In a specific embodiment of the agent, the PMO is modified for intracellular delivery.

Modifications for cellular delivery may include endocytosis-stimulating peptides, such as weak-base amphiphilic peptides taught in U.S. Pat. No. 7,084,248 and commercially available under the tradename ENDO PORTER from Gene Tools, LLC (Philomath, OR, USA). In another example, the PMO is conjugated to an octa-guanidine dendrimer. A specific embodiment of the octa-guanidine dendrimer has the following structure:

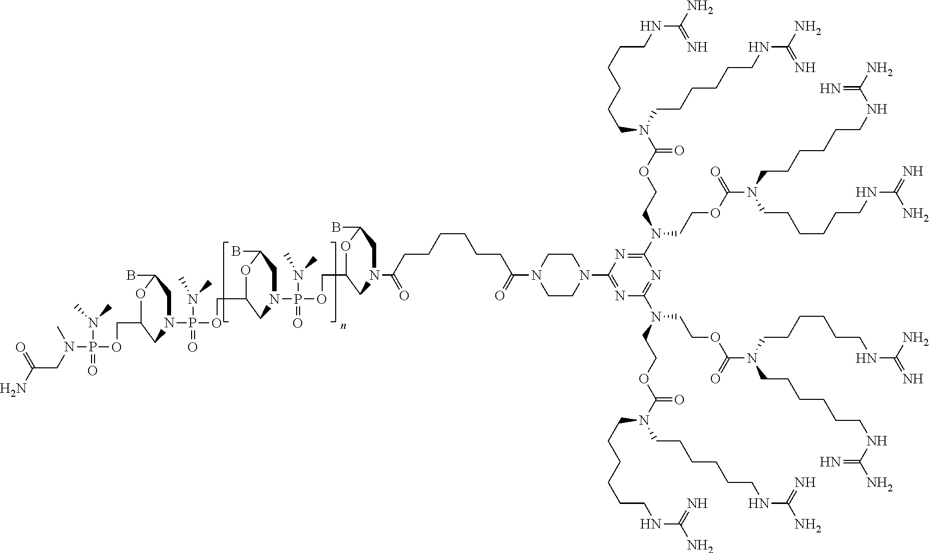

### C. Pharmaceutical Compositions

A pharmaceutical composition for treating or preventing a disease mediated by a strain of a coronavirus (such as SARS-CoV-2) is provided, the composition comprising any of the antiviral agents provided above. The compositions disclosed may comprise one or more of such antiviral agents, in combination with a pharmaceutically acceptable carrier. Examples of such carriers and methods of formulation may be found in Remington: The Science and Practice of Pharmacy (20th Ed., Lippincott, Williams & Wilkins, Daniel Limmer, editor), and are generally well understood by those skilled in the art. To form a pharmaceutically acceptable composition suitable for administration, such compositions will contain a therapeutically effective amount of an antiviral agent.

The pharmaceutical compositions of the disclosure may be used in the treatment and prevention methods of the present disclosure. Such compositions are administered to a subject in amounts sufficient to deliver a therapeutically effective amount of the antiviral agent so as to be effective in the treatment and prevention methods disclosed herein. The therapeutically effective amount may vary according to a variety of factors such as the subject's condition, weight, sex, and age. For example, some embodiments of the composition comprise up to the median lethal dose (LD₅₀) of the antiviral agent. The LD₅₀ can be ascertained using standard toxicological methods, or by reference to past studies. Alternatively, the pharmaceutical composition may be formulated to achieve a desired concentration of the antiviral agent at the site of the infection.

The toxicities of PMOs are generally very low. In some embodiments of the pharmaceutical composition, the PMO is administered to the subject in an amount of up to 0.05, 0.1, 0.15, 0.2, 0.3, 0.5, 1, 1.5, 2, 3, 5, 10, 15, 20, 30, 50, 100, 200, and 500 mg/kg, about any of the foregoing values, and a range between any of the foregoing values. The PMO may be administered to the subject, such as in a pharmaceutical composition, to provide the PMO at a dosage/body mass concentration of up to an amount selected from: 1, 0.05, 0.1, 0.15, 0.2, 0.3, 0.5, 1, 1.5, 2, 3, 5, 10, 15, 20, 30, 50, 100, 200, and 500 mg/kg, about any of the foregoing values, and a range between any of the foregoing values. mg/kg, about any of the foregoing values, and a range between any of the foregoing values.

Other factors include the mode and site of administration. The pharmaceutical compositions may be formulated to be provided to the subject in any method known in the art. Exemplary dosage forms include ocular, subcutaneous, intravenous, topical, epicutaneous, oral, intraosseous, intramuscular, intranasal, and pulmonary. The compositions of the present disclosure may be formulated to be administered only once to the subject or more than once to the subject. Furthermore, when the compositions are administered to the subject more than once, they may be formulated for a variety of regimens, such as once per day, once per week, once per month, or once per year. The compositions may also be formulated to be administered to the subject more than one time per day. The therapeutically effective amount of the antiviral agent and appropriate dosing regimens may be identified by testing in order to obtain optimal activity while minimizing any potential side effects. In addition, a formulation for co-administration or sequential administration of other agents may be desirable.

The compositions of the present disclosure may be formulated to be administered systemically, such as by intravenous administration, or locally such as by subcutaneous injection or by application of a gel, fiber, paste, or cream.

The compositions of the present disclosure may further comprise agents which improve the solubility, half-life, absorption, etc. of the antiviral agent. Furthermore, the compositions of the present disclosure may further comprise agents that attenuate undesirable side effects and/or decrease the toxicity of the antiviral agent. Examples of such agents are described in a variety of texts, such as Remington: The Science and Practice of Pharmacy (20th Ed., Lippincott, Williams & Wilkins, Daniel Limmer, editor).

The compositions of the present disclosure can be formulated in a wide variety of dosage forms for administration. For example, the compositions can be in the form of tablets, capsules, sachets, lozenges, troches, pills, powders, granules, elixirs, tinctures, solutions, suspensions, syrups, ointments, creams, pastes, emulsions, or solutions for intravenous administration or injection. Other dosage forms include for administration transdermally, via patch mechanism or ointment. Further dosage forms include formulations suitable for delivery by nebulizers or metered-dose inhalers. Any of the foregoing may be modified to provide for timed-release and/or sustained-release formulations.

In the present disclosure, the pharmaceutical compositions may further comprise a pharmaceutically acceptable carrier. Such carriers may include vehicles, adjuvants, surfactants, suspending agents, emulsifying agents, inert fillers, diluents, excipients, wetting agents, binders, lubricants, buffering agents, disintegrating agents, accessory agents, coloring agents, and flavoring agents (collectively referred to herein as a carrier). Typically, the pharmaceutically acceptable carrier is chemically inert to the antiviral agents and has no detrimental side effects or toxicity under the conditions of use. The pharmaceutically acceptable carriers can include polymers and polymer matrices. The nature of the pharmaceutically acceptable carrier may differ depending on the particular dosage form employed and other characteristics of the composition.

For instance, in compositions for oral administration in solid forms, such as tablets, capsules, sachets, lozenges, troches, pills, powders, or granules, the antiviral agent may be combined with an oral, non-toxic pharmaceutically acceptable inert carrier, such as inert fillers, suitable binders, lubricants, disintegrating agents, and accessory agents. Suitable binders include, without limitation, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include, without limitation, sodium oleate, sodium stearate, magnesium stearate, sodium benzoate, sodium acetate, and the like. Disintegrators include, without limitation, starch, methyl cellulose, agar, bentonite, xanthan gum, and the like. Tablet forms can include one or more of the following: lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, as well as the other carriers described herein. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, and gels containing, in addition to the active ingredient, such carriers as are known in the art.

The composition may also be in oral liquid forms, such as a tincture, solution, suspension, elixir, and syrup; and the antiviral agents of the present disclosure can be dissolved in diluents, such as water, saline, or alcohols. Furthermore, the oral liquid forms may comprise suitably flavored suspending or dispersing agents such as synthetic and natural gums, for example, tragacanth, acacia, methylcellulose, and the like. Moreover, when desired or necessary, suitable coloring agents or other accessory agents can also be incorporated into the mixture. Other dispersing agents that may be employed include glycerin and the like.

Formulations suitable for parenteral administration include aqueous and non-aqueous, isotonic sterile injection solutions, which can contain anti-oxidants, buffers, bacteriostats, and solutes that render the formulation isotonic with the blood of the patient, and aqueous and non-aqueous sterile suspensions that can include suspending agents, solubilizers, thickening agents, stabilizers, and preservatives. The composition may comprise a physiologically acceptable diluent, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap, an oil or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include polyethylene sorbitan fatty acid esters, such as sorbitan monooleate and the high molecular weight adducts of ethylene oxide with a hydrophobic base, formed by the condensation of propylene oxide with propylene glycol, oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include: (a) cationic detergents such as, for example, dimethyldialkylammonium halides, and alkylpyridinium halides; (b) anionic detergents such as, for example, alkyl, aryl, and olefin sulfonates, alkyl, olefin, ether, and monoglyceride sulfates, and sulfosuccinates; (c) nonionic detergents such as, for example, fatty amine oxides, fatty acid alkanolamides, and polyoxyethylene polypropylene copolymers; (d) amphoteric detergents such as, for example, alkylbeta-aminopropionates, and 2-alkylimidazoline quaternary ammonium salts; and (e) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17.

Topical dosage forms, such as ointments, creams, pastes, and emulsions, containing the antiviral agent, can be admixed with a variety of carrier materials well known in the art, such as, e.g., alcohols, aloe vera gel, allantoin, glycerine, vitamin A and E oils, mineral oil, PPG2 myristyl propionate, and the like, to form alcoholic solutions, topical cleansers, cleansing creams, skin gels, skin lotions, and shampoos in cream or gel formulations. Inclusion of a skin exfoliant or dermal abrasive preparation may also be used. Such topical preparations may be applied to a patch, bandage, or dressing for transdermal delivery, or may be applied to a bandage or dressing for delivery directly to the site of a wound or cutaneous injury.

The antiviral agents of the present disclosure can also be formulated to be administered in the form of liposome delivery systems, such as small unilamellar vesicles, large unilamellar vesicles, and antiemetics. Liposomes can be formed from a variety of phospholipids, such as cholesterol, stearylamine, or phosphatidylcholines. Such liposomes may also contain monoclonal antibodies to direct delivery of the liposome to a particular cell type or group of cell types.

The antiviral agents of the present disclosure may also be coupled with soluble polymers as targetable drug carriers. Such polymers can include polyvinyl-pyrrolidone, pyran copolymer, polyhydroxypropylmethacryl-amidephenol, polyhydroxyethylaspartamidephenol, or polyethyleneoxidepolylysine substituted with palmitoyl residues. Furthermore, the antiviral agents of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates, and cross-linked or amphipathic block copolymers of hydrogels.

### D. Methods of Use

By way of non-limiting example only, methods of using the antiviral agents and pharmaceutical compositions disclosed above are provided.

A method of treatment or prevention of a disease mediated by a coronavirus, such as SARS-CoV-2, in a subject in need thereof is provided, the method comprising administering to the subject a therapeutically effective amount of any of the pharmaceutical compositions or antiviral agents disclosed above. The disease may be any that is caused, complicated, or exacerbated by a coronavirus infection, which in the case of SARS-CoV-2 infection, includes COVID-19. The coronavirus infection need not be in the subject him or herself; for example, the method could be used for the prevention of microcephaly in a fetus by the administration to the mother.

The method of treatment and/or prevention comprises administering to the subject the antiviral agent or the pharmaceutical composition in an amount sufficient to treat or prevent the coronavirus-mediated disease (i.e., a therapeutically effective amount). The method will often further comprise identifying a subject in need of such treatment or prevention. Too little antiviral agent would fail to provide the therapeutic effect. On the other hand, an excessive amount of antiviral agent could lead to undesired side effects.

The therapeutically effective amount may vary according to a variety of factors such as the subject's condition, weight, sex, and age. For example, some embodiments of the method comprise the administration of up to the median lethal dose (LD₅₀) of the antiviral agent. The LD₅₀ can be ascertained using standard toxicological methods, or by reference to past studies. Alternatively, the method may comprise delivering a desired concentration of the antiviral agent to a tissue, organ, or cell type that hosts the coronavirus (such as SARS-CoV-2) in the subject.

If, after the administration of the antiviral agent, the subject still has the coronavirus-mediated disease, or is at risk for the same, then an optional step of the method is to continue the administration of the antiviral agent or pharmaceutical composition.

In one embodiment, the method comprises delivering the antiviral agent to a tissue, organ, or cell type of the subject that hosts a strain of coronavirus (e.g., SARS-CoV-2). Such tissues and organs include the lungs, respiratory epithelial cells, endothelial cells, circulatory system organs or cells, respiratory system organs or cells, cardiac system pulmonary system organs or cells, renal system organs or cells, gastrointestainl system organs or cells, lung cells, brain cells, skin cells, podocytes, monocytes, T lymphocytes, macrophases carbomyocytes, eyes, retinal tissue, retinal endothelial cells, retinal microvascular endothelial cells, retinal pigmented epithelial cells, retinal pericytes, kidney, glomerular tissue, glomerular podocytes, renal glomerular endothelial cells, mesangial cells, cytotrophoblasts, syncytiotrophoblast, human brain microvascular endothelial cells, human neural stem cells, astrocytes, neuroblastoma cells, neural progenitor cells, placental endothelial cells, placental fibroblasts, Hofbauer cells, amniotic epithelial cells, chorionic villi cells, keratinocytes, dermal fibroblasts, dendritic cells, umbilical vein endothelial cells, aortic endothelial cells, coronary artery endothelial cells, saphenous vein endothelial cells, glial cells, primary spermatocytes, Sertoli cells, retinal bipolar cells, retinal ganglion cells, optic nerve cells, and Vero cells, and any combination of the foregoing. It is desirable to deliver the antiviral agent to such targets because they are the sites of infection and replication. Targeted delivery could also prevent unwanted effects on other tissues or organs. In an alternate embodiment, the method comprises administering the antiviral agent locally, such as to the subject's respiratory system (e.g., nasally).

A method of reducing or preventing the replication of a strain of a coronavirus (e.g., SARS-CoV-2) in a host cell is provided, the method comprising contacting the host cell with an effective concentration any of the antiviral agents described above. In a specific embodiment of the method, the effective concentration is at least about 10, 12, 15, 20, 30, 50, 100, 250, or 500 μM. In a further specific embodiment of the method, the effective concentration is about 10, 12, 15, 20, 30, 50, 100, 250, or 500 μM, or any subrange thereof. The host cell may be situated in vivo or ex vivo and may be any cell type known to be permissive to coronaviruses, including any of those listed above.

A method of controlling the spread of coronaviruses in donated tissue is provided, the method comprising exposing the donated tissue to an effective amount of any embodiment of the antiviral agent or pharmaceutical composition disclosed above. The donated tissue may be in the form of a donated organ. The organ or tissue may be exposed to the antiviral agent by perfusing the organ or tissue with a solution containing the effective concentration of the antiviral agent. In a specific embodiment of the method, the effective concentration is at least about 10, 12, 15, 20, 30, 50, 100, 250, or 500 μM. In a further specific embodiment of the method, the effective concentration is about 10, 12, 15, 20, 30, 50, 100, 250, or 500 μM, or any subrange thereof. The antiviral agent may be part of an organ preservation composition, such as the University of Wisconsin cold storage solution (available from Bridge to Life Ltd., Columbia, South Carolina) or any other organ preservation solution known in the art. Another aspect of the disclosed work is a treated donated organ or tissue, comprising an organ preservation composition that includes an effective amount of any of the antiviral agents listed above.

### E. Prophetic Example 1

The use of PMO based technology targeting the nucleotide translation initiation complex site of ZIKV for antiviral development will be explored.

The 5′UTR sequences of SARS-CoV-2 are similar to those of other SARS-like-βCoVs with nucleotide identities of >83.6% [1]. The approach of targeting highly conserved sequences among βCoVs in both human and animal viruses with specific morpholinos should provide broad protection against phylogenetically similar family member βCoVs. The initial approach is to design a morpholino that will anneal to 5′UTR to accomplish translation arrest post-infection to inhibit the production of all viral proteins downstream including the pp1a protease that is required for viral replication and thereby producing no viral progeny and no viral proteins. The synthesis of the morpholino will be performed by Gene Tools. In vitro toxicity will be examined in lung adenocarcinoma cell line A549 and the lung epithelial cell line Calu-3. In collaboration with Ann Eakin, Ph.D. Senior Scientific Officer, Concept Acceleration Program Office of Biodefense, Research Resources & Translational Research/DMID/NIAID/NIH, SARS-CoV-2 will be cultivated in HCT-8 [HRT-18] ATCC® CCL-244 cells according to the manufacturer's recommendations. SARS-CoV-2 infection of permissive cells and controls will be performed in triplicate in 6 well dishes pretreated with 0.1 uM-10 uM of the antiviral agent (referred to as “MRCV-19” in these prophetic examples) reconstituted in purified water to determine the effect and effective dose for MRCV-19 on SARS-CoV-2 replication. qRT-PCR and western blot analysis will be performed to determine the effects of MRCV-19 treatment on mRNA and total protein expression of SARS-CoV-2 envelope and spike glycoproteins. The effects MRCV-19 on SARS-CoV-2 induction proinflammatory cytokines will also be determined [2]. It will also be determined whether MRCV-19 can prevent SARS-CoV-2-induced death in newborn mice. It is expected that significant suppression of SARS-CoV-2 mRNA, protein expression, and induction of proinflammatory cytokines will occur. It is also expected that a significant increase in survival of newborn C57BL/6 mice pups inoculated intracerebrally [3] with SARS-CoV-2 after treatment with MRCV-19 compared to controls will occur. MRCV-19 effectiveness to inhibit COVID-19 replication and gene expression in culture and in a mouse infection model will be done via the DMID/NIAID/NIH collaboration.

Selecting a 5′ UTR target sequence based on conserved sequences among SARS-CoV, SARS-CoV-2 (COVID-19), and the bat SARS-like-CoVZXC21 that represent both human and animal sequences that are most similar to the SARS-CoV-2 virus that causes COVID-19. The sequences represent the 5′UTR for SARS-CoV-2, the bat SARS-like-CoVZXC21 and SARS-CoV [4-6]. The start site for pp1a (ATG) is shown in highlighting with white text (FIG. 1). A complementary sequence that overlaps the 5′UTR and the regions immediately downstream of the ATG start site was identified as the target to engage the SARS-CoV-2 virus upon entry into permissive cells in vitro and in vivo based on its genome structure (FIG. 2A). The structure of the in vivo morpholino and the complementary sequence targeting SARS-CoV-2 genome are shown in FIG. 2B.

The SARS-CoV-2-targeted phosphoramidate dendrimer MRCV-19 will be designed to be complementary to the 25-mer nucleotide sequence within the 5′UTR of the pp1a gene sequence that includes the pp1a ATG start site designated as MRCV-19 start site (FIG. 2A). The control dendrimer reagent used in this study will be a standard control oligo that targets a human beta-globin intron mutation that causes beta-thalassemia. Controls cause little change in phenotype in any known test system except human beta-thalassemia hematopoietic cells and are appropriate negative control for custom phosphoramidate dendrimer.

### REFERENCES

1. Chan J F, To K K, Tse H, et al. Interspecies transmission and emergence of novel viruses: lessons from bats and birds. Trends Microbiol. 2013; 10:544-555.
2. Ye Q, Wang B, Mao J. The pathogenesis and treatment of the ‘Cytokine Storm’ in COVID-19. J Infect. 2020 Apr. 10: S0163-4453 (20) 30165-1. doi: 10.1016/j.jinf.2020.03.037.
3. Antiviral Activity of Chloroquine against Human Coronavirus OC43 Infection in Newborn Mice June 2009 Antimicrobial Agents and Chemotherapy 53 (8): 3416-21.
4. Chan J F, Yuan S, Kok K H, To K K, Chu H, Yang J, Xing F, Liu J, Yip C C, Poon R W, Tsoi H W, Lo S K, Chan K H, Poon V K, Chan W M, Ip J D, Cai J P, Cheng V C, Chen H, Hui C K, Yuen K Y. A familial cluster of pneumonia associated with the 2019 novel coronavirus indicating person-to-person transmission: a study of a family cluster. Lancet. 2020 Feb. 15; 395 (10223): 514-523. doi: 10.1016/S0140-6736 (20) 30154-9. Epub 2020 Jan. 24.
5. Hu D, Zhu C, Ai L, He T, Wang Y, Ye F, Yang L, Ding C, Zhu X, Lv R, Zhu J, Hassan B, Feng Y, Tan W, Wang C. Genomic characterization and infectivity of a novel SARS-like coronavirus in Chinese bats. Emerg Microbes Infect. 2018 Sep. 12; 7 (1): 154. doi: 10.1038/s41426-018-0155-5.
6. Marra M A, Jones S J, Astell C R, Holt R A, Brooks-Wilson A, Butterfield Y S, Khattra J, Asano J K, Barber S A, Chan S Y, Cloutier A, Coughlin S M, Freeman D, Girn N, Griffith O L, Leach S R, Mayo M, McDonald H, Montgomery S B, Pandoh P K, Petrescu A S, Robertson A G, Schein J E, Siddiqui A, Smailus D E, Stott J M, Yang GS, Plummer F, Andonov A, Artsob H, Bastien N, Bernard K, Booth T F, Bowness D, Czub M, Drebot M, Fernando L, Flick R, Garbutt M, Gray M, Grolla A, Jones S, Feldmann H, Meyers A, Kabani A, Li Y, Normand S, Stroher U, Tipples G A, Tyler S, Vogrig R, Ward D, Watson B, Brunham R C, Krajden M, Petric M, Skowronski D M, Upton C, Roper R L. The Genome sequence of the SARS-associated coronavirus. Science. 2003 May 30; 300 (5624): 1399-404. doi: 10.1126/science. 1085953.

### F. Prophetic Example 2—Effective Amounts of “MRCV-19”

A549 cell toxicity assays will be used to test a range of concentrations from 0.1 μM to 10 μM. To determine an effective concentration of MRCV-19 that inhibits SARS-CoV-2 replication, A549 cells, will be pretreated for 24 h with various concentrations of MRCV-19 ranging from 0.1 to 10 UM, rinsed and mock-infected or infected with SARS-CoV-2 at a multiplicity of infection (MOI) of 0.1 in the absence of MRCV-19. Seventy-two hours after infection, the cells will be collected and total protein and intracellular SARS-CoV-2 RNA accumulation will be determined by qRT-PCR and western blot analysis. Anticipated results. MRCV-19 will reduce intracellular SARS-CoV-2 mRNA accumulation in a dose-dependent manner by >95%. Immunoblot analysis will demonstrate a strong inhibition of SARS-CoV-2 protein expression by MRCV-19.

### G. Prophetic Example 3—Preclinical Studies

Animal toxicity studies. MRCV-19 toxicity studies will be performed in CD-1 mice as a fee for service by Pacific Biolab, Hercules CA. Analysis that includes daily animal monitoring of morbidity and mortality 96 hours after subcutaneous injection will be performed. Harvesting of mouse lung tissue from MRCV-19 treated and untreated animals will be done to determine the virus burden, or load, before and after treatment.

Demonstrate MRCV-19 inhibition of SARS-CoV-2 replication in cell culture and in a mouse model. It will be demonstrated that MRCV-19 inhibition of SARS-CoV-2 replication and protein expression occurs in human lung and respiratory epithelial and endothelial cells and cell lines human cerebral organoid model in lieu of Phase I trials. Suppression of SARS-CoV-2 infection with a mouse infection model will be examined. Viral titers post-infection will be examined in lung and respiratory tissue that will be formalin-fixed and paraffin-embedded and analyzed by immunohistochemistry (IHC). In addition, the viral burden will be quantitated using total mRNA extracted from fresh frozen mouse brain tissue by qRT-PCR.

### H. Conclusions

It is to be understood that any of the given elements of the disclosed embodiments of the invention may be embodied in a single structure, a single step, a single substance, or the like. Similarly, a given element of the disclosed embodiment may be embodied in multiple structures, steps, substances, or the like.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and is capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein. Any section headings herein are provided only for consistency with the suggestions of 37 C.F.R. § 1.77 or otherwise to provide organizational queues. These headings shall not limit or characterize the invention(s) set forth herein.

1.-21. (canceled)

22. An antiviral agent that restricts the replication of a coronavirus in a cell, comprising:
a phosphorodiamidate morpholino oligomer (PMO) comprising an antisense sequence to a 5′ portion of a genome of a strain of coronavirus, wherein the 5′ portion comprises the pp1a gene region and the untranslated region (UTR).

23. The antiviral agent of claim 22, wherein the antisense sequence is 5′-AGG GAC AAG GCT CTC CAT CTT ACC T-3′ (SEQ ID NO: 1) or 5′-AAG AAC AAG GCT CTC CAT CTT ACC T-3′ (SEQ ID NO: 3).

24. The antiviral agent of claim 22, wherein the strain of coronavirus is selected from the group consisting of SARS-CoV-2, SARS-CoV, and bat SARS-like-CoVZXC21.

25. The antiviral agent of claim 22, wherein the agent comprises a moiety for intracellular delivery.

26. The antiviral agent of claim 22, wherein the agent comprises an octaguanidine dendrimer delivery moiety.

27. The antiviral agent of claim 22, wherein the agent comprises an octaguanidine dendrimer of the following structure:

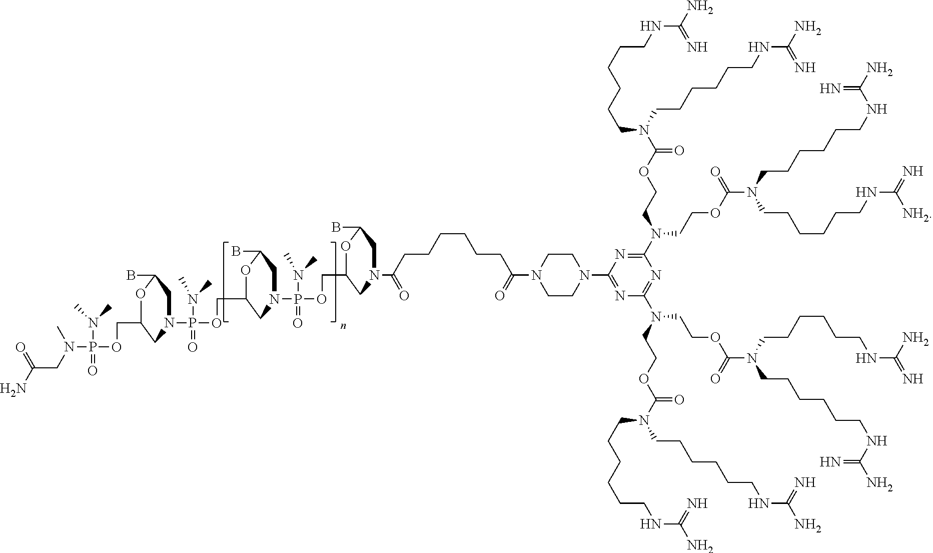

28. An antiviral agent that restricts the replication of a coronavirus in a cell, comprising:
a phosphorodiamidate morpholino oligomer (PMO) comprising an antisense sequence to a 5′ portion of a genome of SARS-CoV-2, wherein the 5′ portion comprises the pp1a gene region and the untranslated region (UTR) and the antisense sequence is 25 nucleotides in length.

29. The antiviral agent of claim 28, wherein the antisense sequence is 5′-AGG GAC AAG GCT CTC CAT CTT ACC T-3′ (SEQ ID NO: 1) or 5′-AAG AAC AAG GCT CTC CAT CTT ACC T-3′ (SEQ ID NO: 3).

30. The antiviral agent of claim 28, wherein the agent comprises a moiety for intracellular delivery.

31. The antiviral agent of claim 28, wherein the agent comprises an octaguanidine dendrimer delivery moiety.

32. The antiviral agent of claim 28, wherein the agent comprises an octaguanidine dendrimer of the following structure:

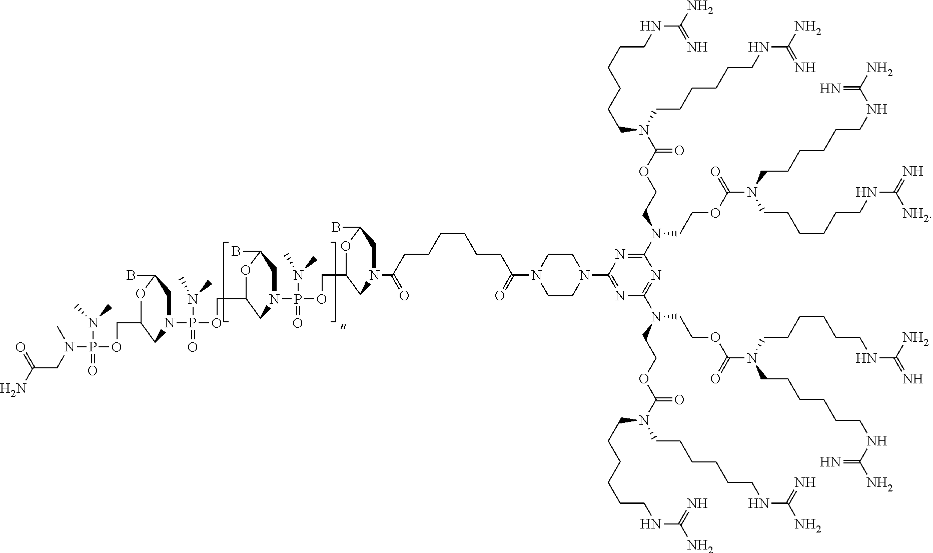

33. A method of reducing the replication of a coronavirus in a host cell in vitro, the method comprising contacting the host cell in vitro with an effective amount of the antiviral agent of claim 22.

34. The method of claim 33, wherein the host cell is selected from the group consisting of a respiratory epithelial cell, an endothelial cell, a retinal endothelial cell, a retinal microvascular endothelial cell, a retinal pigmented epithelial cell, a retinal pericyte, a kidney cell, a glomerular podocyte, a renal glomerular endothelial cell, mesangial cell, cytotrophoblasts, syncytiotrophoblast, human brain microvascular endothelial cells, human neural stem cells, astrocytes, neuroblastoma cells, neural progenitor cells, placental endothelial cells, placental fibroblasts, Hofbauer cells, amniotic epithelial cells, chorionic villi cells, keratinocytes, dermal fibroblasts, dendritic cells, umbilical vein endothelial cells, aortic endothelial cells, coronary artery endothelial cells, saphenous vein endothelial cells, glial cells, primary spermatocytes, Sertoli cells, retinal bipolar cells, retinal ganglion cells, optic nerve cells, Vero cells, and combinations thereof.

35. The method of claim 33, wherein the host cell is selected from the group consisting of: a respiratory epithelial cell, an endothelial cell, and a combination thereof.

36. A method of reducing the replication of a coronavirus in a host cell in vitro, the method comprising contacting the host cell in vitro with an effective amount of the antiviral agent of claim 28.

37. The method of claim 36, wherein the host cell is selected from the group consisting of: a respiratory epithelial cell, an endothelial cell, and a combination thereof.

38. The method of claim 36, wherein the effective amount is sufficient to provide the antiviral agent at a concentration of at least about 10 μM.

39. The method of claim 36, wherein the effective amount is sufficient to provide the antiviral agent at a concentration of about 10 μM to about 500 μM.


SEQUENCE LISTING (7 sequences)

SEQ ID NO: 1 - DNA, 25 bp, synthetic construct
  misc_feature 1..25: note = Synthetic Phosphorodiamidate Morpholino Oligomer
  source 1..25: mol_type = other DNA
agggacaaggctctccatcttacct

SEQ ID NO: 2 - DNA, 25 bp, Severe acute respiratory syndrome coronavirus 2
  source 1..25: mol_type = genomic DNA
aggtaagatggagagccttgtccct

SEQ ID NO: 3 - DNA, 25 bp, synthetic construct
  misc_feature 1..25: note = Synthetic Phosphorodiamidate Morpholino Oligomer
  source 1..25: mol_type = other DNA
aagaacaaggctctccatcttacct

SEQ ID NO: 4 - DNA, 25 bp, Severe acute respiratory syndrome coronavirus 2
  source 1..25: mol_type = genomic DNA
gctgggaaagaccagagactccatg

SEQ ID NO: 5 - DNA, 29891 bp, Severe acute respiratory syndrome coronavirus 2
  source 1..29891: mol_type = genomic DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagtcagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttacttattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatctacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgttcaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagcttttggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaa

SEQ ID NO: 6 - DNA, 29727 bp, SARS coronavirus Urbani
  source 1..29727: mol_type = genomic DNA
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagttcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgtttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgctactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggataagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggagctcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttggatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacaccccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatatgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgac

SEQ ID NO: 7 - DNA, 29732 bp, Bat SARS-like coronavirus
  source 1..29732: mol_type = genomic DNA
atattaggtttttaccttcccaggtaacaaaccaactaactctcgatctcttgtagatctgttctctaaacgaactttaaaatctgtgtgactgtcacttagctgcatgcttagtgcactcacgcagtttaattataattaattactgtcgttgacaggacacgagtaactcgtctatcttctgcaggttgcttacggtttcgtccgtgttgcagccgatcatcagcataccttggtttcgtccgggtgtgaccgagaggtaagatggagagccttgtccctggtttcaacgagaaaacacacgtccaactcagtttgcctgttttacaggtttgtgacgtgcttgtacgtggctttggagactccgtagaggaggctttatcagaggcacgtcaacatcttaaagatggcacttgtggcttagtagaagttgaaaaaggtgttttacctcaacttgaacagccctatgtgttcatcaaacgttctgatgcccgaactgcacatcatggccatgttatggtcgaattagtagcagaactcgatggcattcagtacggtcgtagtggtgagacacttggtgttcttgtccctcatgtgggagaggtaccagttgcttaccgtaaagttcttcttcgtaagaacggtaataaaggagctggtggccatagttacggcgccgatctaaagtcttttgacttaggcgacgagcttggtactgatcctattgaagattttcaagaaaattggaacactaaacatggcagtggtgttacccgtgaactcaagcgtgagcttaacgggggtgcatacactcgctatgtagacaacaacttttgtggcccagatggctaccctcttgagtgcattaaaaactttttagctcgtgctggtaaggcttcatgcactttatctgaacaactggattttattgacactaaaagaggtgtatactgctgccgtgaacatgagcatgaaattgcttggtacacggaacgctccgaaaagagctatgaatcgcagacaccttttgaaattaagttggcaaagaaatttgacaccttcaatggagaatgtccaaattttgtatttcctctaaattcaacaatcaagaccattcaaccaagggttgaaaagaaaaagcttgatggtttcatgggtagaattcgatctgtctatcctgttgcttcaccaaatgaatgcaaccaaatgtgcctctcaactctcatgaagtgtgaccattgtggtgaaacttcatggcagacgggtgattttgttagagccacttgcgaattctgtggtactgaaaatttgactaaagaaggcgccacaacttgtggttacttacctcaaaatgctgttgtaaaaatttattgtccagcatgtcataatccagaagtgggacctgagcatagtcttgctgaatatcataacgagtctggtttgaaaaccgttcttcgtaagggtggtcgtaccattgcttatgggggctgtgtgtttgcttatgttggttgctacaacaagtgtgcctattgggttccacgtgctagtgctaacataggctgtaatcacacaggtgttgttggagaaggttctgaaagtctaaacgacaaccttcttgaaatattgcaaaaggagaaagtcaacatcaatattgttggtgacttcaaacttaatgaagagattgccattattttggcatctttttctgcttctacaagtgcttttgtagaaactgtaaaaggtttggattacaaaacattcaaacaaattgttgaatcctgtggtaactttaaagttactaaaggaaaggctaagaagggtgcctggaacattggggaacagaaatcaatactgagtcctctttatgcatttgcatcagaggctgctcgtgttgtgcgctccattttttctcgtactcttgaaactgctcaatactctgtgcgtgtcttacaacaggccgccataacaatcttagatggaatttcacagtattccctgagactcattgatgctatgatgttcacatctgacttggttactaacaatctagttgtaatggcttacattacgggtggtgttgtacaaatgacttcacagtggctaacaaatatctttggcactgtttatgaaaaacttaaaccggttcttgattggctcgaagagaaattcaaggaaggtgtagagtttcttagagacggttgggaaattgttaaatttatctctacttgtgcttgtgaaattgtcggtggacaaattgtcacccgtgcaaaggaagttaaggagagtgttcagacattctttaagcttgtaaataaatttttggctttgtgtgctgactccatcattattggtggagctaaacttaaagccttgaatttaggtgaaacatttgtcacacactcaaagggattgtacagaaagtgtgttagatccagagaagaaactggcttactcttgcctctgaaagccccaaaagaaattattttcttagagggagaaacacttcccacagaagtgttaacagaggaagttgtcttgaaaactggtgttttacaaccattagaacaacctactaatgaggctgttgaagccccattgattggtacaccagtctgtattaacgggctcatgttgctcgaaattaaagacacagaaaagtactgtgcccttgcacctaatatgatggtaacaaataatacctttacacttaaaggcggtgcaccaacaaaagtcacttttggtgatgacactgtgattgaagtgcagggttacaagagtgtaaacatcacttttgaacttgatgaaaggattgataaagtacttaatgagaagtgctctagttatacagttgaactcggtacagatgtaaacgagttcgcttgtgttgttgctgatgctgtcataaaaactttacaaccagtatctgaactactcacaccactgggcattgatttagacgagtggagtatggctacatactacttgtttgatgagtccggtgaatttaaattgtcttcacatatgtactgttctttttaccctcctgaagatgaaggggaagatgattgtgaagaaggacagtttgaaccatcaactcaatatgagtatggtactgaggatgactaccaaggtaaacctttggagtttggtgctacttctttttcttcttcttcacaggaagaagaacaagaagaggattggttagaatctgatagtcaggacggccaagagactgctgttactaaaactagtgaacaaaataatttcacaggttatttaaaattaactgacaatgttttcattaaaaatgctgacattgtagaagaagctaaaaaggtaaagcctacagtagttgttaatgcagctaatatttaccttaaacatggaggaggtgttgctggagctttaaataaagcaactaacaacgccatgcagttgaatctgattagtaacataactaccaatgggccactaattgtgggtggtagttgtgttttgagtggacataaccttgctaaaaattgtcttcatgttgttggccctaatgtcaatagaggtgaagacattcaattgcttaaaaatgcttatgaaaatttcaatcaacatgagattttactcgcaccattattatcggctggtatttttggtgttgatcctgtacattctttaagagtttgtgtagaaactgttcacacaaatgtctatctagttgtctttgacaaaaatctttatgacaaacttgtttcaagctttttagagatgaagagtggaaaacaagtagaacaaaaagttgctgaaaatcctaaagaggaagttaagccattttttactgaaaataaaccttcagttgaacaaagacaacaagctgaagagaagaaaatcaaagctagtattgaagaagttacaactactctagaggagaccaagttccttacagaaaacttgttactttatattgacatcaatggcaatcttcacccagattctgccactcttgttaaagatattgacatcactttcttgaagaaagatgttccctatatagtgggtgatgttattaaagaaggtgctttaactgctgtagttatacctactaaaaaggctggtggcactactgaaatgcttgctaaagctttaagaaaagtgccaacagataatcacataaccacctaccctggtcagggtttaaatggttacactgtagaagaagcaaagacagtgcttaaaaagtgtaaaagtgttttttacattttaccatctattctccctaatgagaagcaagaaattctaggaactgtttcttggaacttgcgagagatgctcgcacatgcagaagaaacacgtaagttaatgcctgtttgtatggagactaaagctatagtttcaactatacaacgtaagtacaaaggcattaaaatacaggagggtgtggttgactatggtgctagattttacttttatactagtaaaactactgtagcatcacttattaacacacttaacaatctaaatgagactcttgtcacaatgccattaggatatgtgacgcacggtctaaatttagaagaagctgcgcggtacatgaggtctctcaaagtaccggctacagtctctgtttcttcaccagatgctgttacagcatataatggttatcttacttcttcttcaaaaacacctgaagaacactttatcgaaaccgtctcacttgctggttcctataaagattggtcttattctggacagtctacagaactaggtatagaatttcttaagagaggtgataagagtgtatattacaccagtaatcctatcacattctatctagatggtgaagttatcacctttgataatcttaagacaattctctctttgagggaagtgaggtctattaaagtgtttacaacagtagataacattaatctccacactcaaattgtggatatgtctatgacatatgggcaacagttcggcccaacttatttggatggagctgatgttactaaaataaaacctcataattcacatgaaggtaaaacattttatgttttgcctaatgatgacactttacgtgtggaggcttttgagtactaccatacaactgactctagttttcttggtaggtacatgtcagcattaaaccacactaaaaagtggaaatacccacaagtaaatggtttaacttctataagatgggcagacaacaattgttatcttgctactgcattattaacacttcaacaaatagagttgaaatttaatccaccagctttacaagatgcctattatagggcaagagctggtgaggctgctaatttttgtgcacttatcctagcctattgtaataagacagtaggtgagttaggtgatgtcagagaaacaatgaattatttgtttcaacatgccaatttagattcttgtaaaagagtcttgaatgtggtgtgtaaaacttgtggacaacagcaaacaactcttaagggtgtagaagctgttatgtatatgggcacactttcttatgaacaacttaagaagggtgtgcagataccttgtatgtgtggtaaacaagctacacaatatctagtacaacaagagtcaccttttgttatgatgtctgcaccacccgcccaatatgaacttaagcatggtacatttgtttgtgctagtgagtatactggtaattaccagtgtggtcactacaaacatataacttctaaagaaaccttgtattgtatagatggtgctttactcacaaagtcctctgagtacaaaggttctattacagatgttttctataaggaaaacagttatacaacaaccataaaaccagttacatacaagttggatggtgttgtttgtacagaaattgatcctaagttggatggttattataagaaagacaattcttatttcacagagcaaccaattgatcttgtaccaaaccaaccttacccgaatgcaagctttgacaattttaggtttgtatgtgataatatcaaatttgccgatgatttaaatcaattgtctggttataagaaacctgctttgagagagcttaaggttacattctttcctgacttaaatggtgatgtagtggctattgattataagcactacacaccttcttttaagaaaggagctaaattgttgcataagccaattgtttggcatgttaacaatgcaactaacaaagcaacgtataaaccaaatatttggtgcatacgttgtctttggagtacaaaaccggttgaaacatcaaattcttttgatgcactgaaattaggggacacacagggaatggataatcttgcctgtgaagttctaaaaccagtctctgaagaagtagtggaaaatcctaccatacagaaagacattcttgagtgtaatgtgaaaactaccgaagttgtaggagacattatacttaaaccggcaattgatggtctaaaaattacagaagaggttggtcatacagacctaatggctgcttatgttgacaattcaagtcttactattaagaaacctagtgaattatccagagtattaggtttgaaaactttagccactcatggcttggctgctattaatagtgttccttgggacactatagctaattatgctaagcctttccttaataaggttgttagcacaactactaacatagtcacacggtgtctaaaccgtgtttgtactaattatatgccttatttgtttactttattgctacaattgtgtacttttactaaaagtacaacttctagaataagagcatctatgccaaccactatagcaaagaatactgttaaaagtgttggtaaattttgtatagaggcttcatttaattatttgaagtcacctaatttttctaaattgataaatattgtaatttggtttttattattaagtgtttgcctaggttctttaatctattcaactgctgctttaggtgtcttaatgtctaatttaggcatgccttcttattgtactgtttacagagaaggttacttgaactctactaacgtcactattgcaacctactgcactggttctataccttgtagtgtttgtcttagtggtttagattctttggatacttacccatccttagaaactatacaaattaccatttcgtcttttaaatgggatttaactgcttttggtctagttgcagagtggtttttggcatatattctttttactaggttcttttatgtacttggattggctgcaatcatgcaattgtttttcagctattttgcagtacattttattagtaattcttggcttatgtggttaataattaatcttgtacaaatggccccaatttcagctatggttagaatgtatattttctttgcatcattttattatgtatggaaaagttatgtgcatgttgtagatggttgtacttcatcaacttgtatgatgtgttataaacgtaatagagcaacaagagttgaatgtacaactattgttaatggtgttagaaggtccttttatgtctatgctaatggaggtaaaggcttttgcaaactacacaactggaattgtatcaattgtgatacattctgtgctggtagtacatttattagtgacgaagttgctagagacttatcactacagtttaaaagaccaataaatcctactgaccagtcttcctatattgttgatagtgttacagtgaagaatggttccatccacctttactttgataaggctggtcaaaagacttatgaaagacattctctctctcattttgttaacttagacaatctgagagctaataacactaagggttcattgcctattaatgttatagtttttgatggtaaatcaaaatgtgaagaatcatctgctaaatcagcgtctgtttattatagtcagcttatgtgccaacctatactgttactggaccaggcattagtgtctgatgttggtgacagtgcagaagttgcagttaagatgtttgatgcttatgttaatatattttcatcaactttcaatgttccaatggaaaaactcaaagcattagttgcgactgcagaagctgaacttgcaaagaatgtgtctttagacaacgtcttatctacttttatttcagcagctcgccaagggtttgttgattcagatgtagaaaccaaagatgttgttgaatgtcttaaattgtcacaccaatctgacatagaagttacaggtgacagttgtaacaattacatgctcacttataacaaagttgaaaacatgacacctcgggacctcggtgcttgtattgattgtagtgcacgtcatattaatgcacatgtggcaaagagtcataacatagctttgatttggaatgttaaagatttcatgtcattgtctgaacaactacgaaaacaaatacgcagtgctgctaagaagaataacttgccttttagattgacatgtgcaaccactagacaagttgttaatgttgttacaacaaaaatagcacttaggggtggtaaaattgttaacaactggttgaagcagttgattaaggttacacttgtgtttcttttcatcactgttatcttctatttaataacacctgctcatgtcatgtttaagcacaatgacttttcaagtgaaattataggatacaaggctattgatggtggtgtcactcgtgacatagcgccaacagatacttgttttgctaacaaacatgctgactttgactcttggtttagtcagcgtggtggtagctatactaatgataaagcttgcccattgatagcagctgttatcacaagagaagttggctttgttgtgcccggtttacctggcacaatattacgcacaattaatggtgacttcttacattttcttcctagagtgtttagtgcggtaggtaacatttgttacactccttctaaacttatagagtacactgactttgcaacatcggcatgtgttttagctgctgaatgtaccatctttaaagattcttctggtaaaccagtgccttattgttatgacactaatgtactagaaggttctgttgcgtatgaaagtctccgccctgacatacgctatgtgctcatggacggttctataattcaattccctaacacctaccttgaaggttctgttagagtagtaacaacttttgattcagagtattgtagacatggtacttgcgaaagatcagaggctggcatttgtgtatctactagtggtagatgggtacttaataatgattattacagatccttgccaggagttttttgtggtgtagatgctgtgaatttacttactaatatgttcacgccattaattcaacctattggtgctttggacatatctgcatctattgtagcaggtggtgttgtagctattatagtaacttgtctagcctactacttcatgaggtttagaagagcttttggtgaatacagtcatgtagttgcctttaacactctactattctttatgtcattcactgtactctgtttaacaccagtctattcattcttacctggtgtttattctgttatttacttgtacttgacattttatcttactaatgatgtttctttcttagcacatatccaatggatggttatgttcacacccttagtgcctttctggatgacaattgtttatgtcatttgcatttccacaaagcatttttattggttctttagtaactacctaaagagacgtgtagtctttaatggtgtttcctttagtacatttgaggaggctgcattatgtacctttttgttaaataaagaaatgtatctgaaattgcgtagtgatgtacttctacctcttacgcaatataatagatatttagctctttataataagtacaagtattttagtggggccatggacactaccagttatagagaagcagcttgctgtcatctggctaaggctctaaatgatttcagtaattcaggttctgatgtcctctaccaaccaccacaaacttcaatcacatcagcgattttgcagagtggttttagaaaaatggcattcccatctggtaaagttgaaggttgcatggtacaagttacttgtggtaccactacacttaatggtctttggcttgatgatgtagtttactgtccacgacatgtgatctgcacttctgaagacatgctcaatcctaattatgaagatttacttatacgtaaatctaaccataattttttagttcaggctggtaatgttcaacttagggttgttggacattctatgcaaaattgtgttcttaagcttaaagtagatacagctaaccctaagacacctaagtataagtttgtgcgcattcaacccggacagactttttcagtattagcctgttacaatggttcaccatctggtgtttaccaatgtgccatgagacctaattttactattaagggttcattccttaatggttcatgtggtagtgttggttttaatatagactatgactgtgtctctttttgttatatgcatcatatggagttaccaacgggagttcatgctggcacagacttagaaggtaccttctacggaccttttgttgacagacagacagcacaagcggctggtactgacacaactattacagttaatgttctagcttggttgtatgcagctgttataaacggagatagatggttccttaacaggtttaccacgactctaaacgattttaatcttgtggctatgaagtataattatgaacctctaacacaagaccatcttgacatactaggacctctttcagctcaaactggaattgcagtcctagatatgtgtgcttcattaaaagaattattacaaaatggtatgaatggacgtaccatattgggtagtgctttattagaagatgaatttacacctttcgatgttgttagacaatgttcaggtgtcacctttcaaagtgcagtgaaaaggacaatcaagggcacgcaccattggttgttgcttacagttttgacttcactcttagttttagttcagagtactcaatggtctttgttcttctttgtgtatgaaaatgcctttatgccttttgctatgggtattattgctatgtctgcttttgctatgatgtttgtcaaacataagcatgcattcctctgtttgttcctattaccttctcttgctactgtagcttattttaatatggtctacatgcctgctagttgggtgatgcgtattatgacatggttggatatagttgatactagtttgtctggtttcaagctaaaggactgtgttatgtatgcatcagctgtagtgttattaatcctcatgacagcaagaactgtatatgatgatagtgctagaagagtttggacacttatgaatgtcctgacactcgtttataaagtttattatggtaatgctttagaccaagctatttccatgtgggctcttataatctctgttacttctaactactcaggtgtagttacaactgtcatgtttttggccagaggtattgtttttatgtgtgttgagtattgtcctatcttctttataactggcaatacactccagtgtataatgctagtttattgtttcttaggttatttctgtacttgttattttggtctcttctgtttactcaaccgctattttaggcttactcttggtgtttatgactatttagtctctacacaagaatttaggtatatgaactctcagggacttctgcctcctaagagtagtattgatgctttcaagcttaatattaaattattgggtattggaggtaagccatgcattaaggttgctactgtacagtctaaaatgtctgacgtaaagtgcacatctgtggtactgctctctgtacttcaacaacttagagtagagtcatcttctaaattgtgggcacagtgtgtacaactccacaatgatatccttcttgctaaagacacaactgaagcttttgaaaagatggtttctcttctgtctgttttgctatccatgcaaggtgctgtagaccttaacaagttgtgcgaggaaatgctcgacaaccgtgctactcttcaagctattgcttcaaaatttagttctttaccatcatatgccgcttatgcaacagcccaagaggcttatgagcaggctgtagttaatggtgattctgaagttgttcttaaaaagttaaagaaatctttgaatgtggctaaatctgagtttgaccgtgatgctgccatgcaacgcaagttggaaaagatggcggatcagactatgacccaaatgtacaagcaggcaagatctgaagacaagagggcaaaagtaactagtgcaatgcaaactatgcttttcactatgcttagaaaacttgataatgatgcacttaacaacattatcaacaatgcacgtgatggttgtgtaccactcaacatcataccactcacaacagcagccaaactcatggttgttgtgcctgactatggaacctacaagaatacttgtgatggtaacacttttacatatgcatcagcactctgggaaatccagcaagttgttgatgcagatagtaaaattgtgcagcttagtgaaatcaacatggacaactcaccaaatttggcttggcctcttattgtcactgctttaagagctaattcagctgtcaaactacagaacaatgagctgagtccagtagcactacggcagatgtcctgtgcggctggtactacacagacagcttgtactgatgacaacgcacttgcctactataacaattctaagggaggtaggtttgtgctggcattactatcagaccatcaagatctcaaatggtctagatttcctaagagtgatggcacaggtactatttacacagagctggaaccaccttgtaggtttgttacagacacaccaaaaggacctaaagtgaagtacttgtactttattaagggtcttaacaacctaaatagaggtatggtactgggtagtttagctgctacagtacgtcttcaggctggtaatgcgacagaagtgcctgccaactcaactgtgctttctttctgtgccttcgctgttgacccagctaaggcctacaaagattacttggcaagtggtggacaaccaatcacgaattgtgtgaagatgttgtgtacacacactggtactggacaggcaattacggtgacaccagaagccaatatggaccaggagtcctttggtggtgcttcgtgctgtctgtattgtagatgccacattgatcacccaaatcctaagggattctgtgatttgaaaggtaagtatgttcaaatacctaccacttgtgctaatgaccctgtgggttttacacttagaaacacagtctgtaccgtctgtggaatgtggaaaggttatggctgtagttgtgatcaactccgcgaacccatgatgcagtctgcggacgcgtcaacgtttttaaacgggtttgcggtgtaagtgcagcccgtcttacaccgtgcggctcaggcattagcactgatgtcgtttacagggcttttgatatttacaacgagaaagttgctggttttgcaaagttcctaaaaactaattgctgccgcttccaggaaaaggatgaggaaggcaatttattagactcttatttcgtagttaagaggcacacaatgtccaactaccaacatgaagagaatatttataacttggtcaaggagtgtccagctgttgctgttcatgactttttcaagtttagagtagatggtgacatggtaccacatatatcacgtcagcgtttaactaagtacacaatggctgatttagtctatgctctacgtcattttgatgagggcaattgtgacacattaaaagaaatacttgtcacatacaattgttgtgatggtgattatttcaataagaaggattggtacgacttcgtagagaatcctgacatcttacgcgtatacgctaacctaggtgagcgtgtacgtcaagcattattaaagactgtacaattctgcgatgctatgcgcgatgcgggcattgtaggtgtactcacactagataatcaggatcttaatgggaactggtacgatttcggtgatttcgtacaagtagcgccaggttgcggagttcctattgttgactcgtattattcattgttgatgcccattcttactctgacaagggcattagctgctgagtcccatatggatgctgatcttgcaaaaccacttattaagtgggatttgttgaaatatgattttacggaagagagactttgtctcttcgaccgttattttaaatattgggatcagacataccatcccaattgtattaactgtttggatgataggtgtatccttcattgtgcaaactttaatgtcttattttctactgtgtttccgcctacaagttttggaccactagttagaaagatatttgtagatggtgtaccttttgttgtttcaacgggataccattttcgtgagctaggggttgtacataatcaggatgtaaacctacatagctcgcgcctcagttttaaggaacttttagtgtacgctgctgatccagctatgcatgctgcctctggtaatttgttgttagataaacgcactacatgcttttcagtagctgcactaacaaataatgttgcttttcaaactgtcaaacccggtaattttaataaagacttttatgactttgctgtgtctaaaggcttctttaaggaaggaagttctgttgaattaaaacacttcttctttgctcaggatggcaatgctgctatcagtgattatgactattaccgttataatctgccaacaatgtgtgatattacacaactcctattcgtaattgaggttgtcgataaatactttgattgttacgatggtggctgtatcaatgccaaccaagttatcgttaacaatctagacaaatcagccggtttcccattcaacaaatggggtaaggctagactttattatgattcaatgagttatgaggaccaagatgcactgttcgcatacactaagcgtaacgtcatccctacaataactcaaatgaaccttaagtatgccattagtgcaaagaatagagctcgcaccgttgctggtgtctctatctgtagtactatgaccaatagacagtttcaccagaaattattaaagtcaatagccgccactagaggagctactgtagtaattggaacaagtaaattttatggtggctggcataacatgttaaaaactgtttacagtgatgtagaaagtcctcaccttatgggttgggactacccaaaatgtgacagagccatgcctaatatgcttagaatcatggcttccctcgttcttgctcgcaaacatagcacttgttgtaacttgtcacaccgtttttatagattagctaatgagtgtgcacaagtattaagtgagatggtcatgtgtggcggctcattatatgtgaaaccaagtggaacgtcatccggtgatgccacaactgcttatgctaatagtgtgtttaacatctgtcaagcagtaacagctaatgtgaatgcacttctctcaactgatggtaataagattgctgataagtacgtccgcaaccttcaacacagactttatgagtgtctctatagaaatagagacgtggatcatgaattcgtggatgaattttatgcatatttgcgtaaacatttctccatgatgatactctcagatgacgcagtcgtatgctataatagtaattatgcggcacagggtttagtagctagcattaagaactttaaagcagttctttactaccaaaacaatgtgtttatgtctgaggcaaaatgctggactgagactgatcttactaaaggacctcacgaattttgctctcagcatacgatgctagttaaacaaggagatgattatgtgtacctgccttatccagatccatctagaatattaggcgcaggctgttttgtcgatgacatcgtcaagacagatggtacacttatgattgaaaggtttgtgtcattagcaattgacgcctacccacttacaaagcaccctaatcaagagtatgctgatgttttccatttatacttacagtatattaggaaattacatgatgagcttaccggtcacatgttggacatgtactctgtaatgctaactaatgacaacacctcaaggtactgggaacctgagttttacgaagcaatgtacacaccacacacggttttacaagctgtgggtgcgtgtgtattatgtaattcacagacttcacttcgttgcggtgcttgcattaggagacctttcctttgttgtaagtgctgctatgatcatgtcatttcaacatcacataagttagtgttgtctgttaatccctatgtttgcaacgcactaggttgtgatgtcacagatgtgacacaactctacttaggaggtatgagctattattgcaagttacataaaccacccattagttttcctttgtgtgctaatggtcaggtctttggtttgtataaaaacacatgtgtgggcagtgataatgtaactgacttcaatgcaatagcgacatgtgactggactaatgctggcgattatatacttgccaacacttgcacagagagactcaaactttttgcagcggaaacgctcaaagctactgaggaaacatttaaactatcttatggtatagccactgttcgtgaagtactgtcagatagagagctttatctttcatgggaggttggaaaacctagaccaccattgaatagaaactacgtctttactggttaccgcgtgaccaaaaatagtaaagtacagattggagagtatacctttgaaaaaggtgactatggtgatgctgttgtgtacagaggtactacaacatacaaattgaatgttggcgattactttgtgttaacatcacacacagtaatgccactaagtgcaccaacactagtgccacaagagcactatgtgcgaataactggcttataccctacacttaacatctctgatgagttttctagcaatgttgcaaattaccaaaaggtcggtatgcagaagtactccacactccaaggaccacctggtactggtaagagtcactttgctattggacttgccctctactacccatctgctcgcatagtgtatacagcttgctctcatgctgctgttgatgcgctatgtgaaaaggcattaaaatatttgcctatagataagtgtagtagaattatccctgcacgtgcgcgtgtagagtgttttgataaattcaaagtgaattcaaccttagaacagtatgttttctgcactgttaatgctctgcctgaaactactgctgatatagtggtctttgatgaaatttcaatggccactaattatgatttgagtgttgtcaatgctagactacgtgcaaaacactacgtttacattggcgatccagctcaattacctgcaccacgcacattgctaacaaaaggcacacttgaaccagaatatttcaattcagtgtgcagacttatgaaaacaataggtccagacatgttccttggaacttgtcgtcgttgtcctgctgaaattgttgacacagtgagtgctttagtttatgataataagctaaaagcacacaaagagaagtcagcgcaatgctttaagatgttttataagggtgtgattacgcatgatgtgtcatccgcaataaacagaccacaaataggtgtagtaagagaatttcttacacgcaaccctgcttggagaaaagctgtcttcatctcaccatataattcacagaacacagtggcatcaaagattttagggttgccaactcaaactgttgattcttcacagggttctgaatatgactatgtcatattcacgcaaaccactgagactgcacactcttgtaatgtaaaccgctttaatgtggccatcacaagagcaaaaattggcattttgtgcataatgtctgatagagacctttatgacaagctgcaatttacgagtctagaagtaccgcgtcgtaatgtggctactttacaagcagaaaatgtgactggactctttaaggactgtagcaagatcattactggtcttcatccaacacaggcacctacacatcttagtgttgatactaaattcaaaactgaaggactttgtgtcgacataccaggaataccaaaagacatgacctatcgtagactcatctctatgatgggttttaaaatgaattaccaagttaatggttaccctaatatgtttatcacccgtgaagaagctatccgtcacgttcgtgcgtggataggctttgatgttgagggttgtcatgcgactagagatgctgtaggaacaaatctaccactccagttagggttttcaacaggtgttaacctagtgactgtaccaactggctatgttgacactgagaatagcacagaattcaccagagttaatgcaaaacctcctccaggtgatcaatttaagcatcttataccacttatgtacaaaggcttgccctggaacgtggtgcgtattaagattgttcaaatgctcagtgacacactgaaaggattatcagacagagttgtgtttgtcctttgggcgcatggctttgaactcacatcgatgaagtactttgtcaagatcggaccagaaagaacgtgttgtctgtgtgacaaacgcgcgacttgcttctctacttcatctgatacttacgcctgttggaatcactctgtgggctttgactatgtctataacccattcatgattgatgtccagcagtggggttttacaggtaaccttcaaagtaaccatgaccagcactgccaagtgcatggtaatgctcatgtagctagttgtgatgctatcatgactagatgtcttgcagtccatgagtgctttgttaagcgtgttgattggtctgttgaatacccaattattggagatgaactgaagattaacgccgcgtgcagaaaagtacagcatatggttgttaaatctgcattgcttgctgataaattcccagttcttcatgacataggaaacccaaaggctattagatgtgtgccgcaggctgaagtggactggaaattctacgacgctcagccttgcagtgataaagcttataaaatagaagaactcttctactcatatgccacacatcatgacaagttcacagatggtgtctgcttgttttggaactgtaacgttgatcgttacccggctaatgctattgtgtgtaggtttgatactagagtgctttctaatttaaacctaccaggttgtgatggtggtagtttgtatgttaataagcatgcattccacactccagcttttgataagagtgcatttacacatttgaaacaactgcctttcttttattactctgacagtccgtgtgagtctcatggcaaacaggttgtgtcagatattgattatgtcccactaaagtctgctacgtgtattacacgatgcaacttaggtggtgccgtttgtagacatcatgcaaacgagtatagacagtacttggatgcatataatatgatgatttctgctggatttagcctttggatttataaacaatttgatacttacaacttgtggaacactttcaccaagttgcagagtttagaaaatgtggcttataatgttattaacaagggacactttgatggacagagtggtgaagcacctgtgtctatcattaataatgctgtttacactaaggtagatggtattgatgtggagatctttgaaaataagacaacacttcctgttaatgttgcatttgagctttgggctaaacgtaacattaaacctgtgccagagattaaaatactcaataatttgggtgttgatatcgctgctaatactgttatatgggaccacaagagagaagctccagcgcatgtttctacaataggtgtctgtacaatgactgacattgcagagaaacctactgagaatgcttgttcatcactcactgtcttatttgatggtagagttgagggacaggcgaacctttttagaaacgctcgtaatggtgttttaataacagaaggttcagttaagggcttaacaccttcgaaaggacctgcacaggctagtgtcaacggggtcacattaattggagaatcagtaaaaacacagttcaattactttaagaaagtggatggcattattcagcaattgccagaaacttactttactcaaagcagagacttagaggatttcaagcccagatcacaaatggaaactgatttccttgagctcgctatggatgaattcatagaacgatataagctagacggctatgctttcgagcacatcgtttatggagattttagtcatggacaattaggcggacttcatttattgataggactagccaaaaggtcacaggactcactgttaaagctagaggattttattcctatggatagcacagtgaaaaactacttcataacagatgcgcaaacgggttcatctaagtgtgtatgctctgttatcgatcttttacttgatgactttgttgaaataataaagtcacaagatctttcagtggtttcaaaagtagtcaaagttacgattgattatacagaaatttcatttatgctttggtgtaaagatgggcatgtggaaactttttacccaaaattacaatctagccacgcatggcaaccaggtgttgctatgcctaatctttataaaatgcagagaatgttactggaaaagtgtgaccttcaaaattatggtgatagtgctacattgcctaaaggcataatgatgaacgtcgcaaagtatactcaactgtgtcagtatttaaatacacttactttagctgtgccctacaatatgagagttatacactttggcgcaggctctgataaaggagtagcacctggcacagctgttcttagacaatggttgccaactggtacactacttgtcgattctgatttaaatgattttgtttctgatgcagattcaacattaattggtgattgtgcaaccgtacatacggctaataaatgggatctcattattagcgatatgtatgatcctaagactaaaaatgttacaaaagagaatgattccaaagaaggatttttcacttacatttgtggatttatacagcaaaaattagccctcggaggttccgtagctgtaaagataacagaacattcctggaatgccgatctttataagctcatgggatacttcgcatggtggacagcttttgttactaatgttaatgcttcatcctcagaggctttcttaattggttgtaattaccttggcaaaccacgtgagcagatagatggttatgtcatgcatgcaaattacatattttggaggaacacaaatccaatacaattgtcttcctactcattatttgacatgagtaagtttcctcttaaattaagaggtactgctgttatgtcattaaaagatggacaaatcaatgatatgattttgtctcttcttagtaaaggcagacttattgttagagagaataatagagttattatctctagtgatgttcttgttaacaactaaacgaacatgttgtttttcttgtttcttcagttcgccttagtaaactcccagtgtgatttgacaggtagaactccactcaatcccaattatactaattcttcacaaagaggtgtttattaccctgacacaatttatagatctgacacactagtgcttagtcagggttattttcttccattttattccaatgttagctggtattattcattaacaaccaacaatgctgccacaaagaggactgacaaccctatattagatttcaaggacggcatatattttgctgccactgaacactcaaatattgtcaggggctggatctttggaacaactcttgacaacacttctcaatctctcttgatagttaacaatgcaacgaatgttattatcaaggtttgtaattttgacttttgttacgatccctaccttagtggttactatcataacaacaaaacctggagcatcagagaatttgctgtctattccttttatgctaattgtacttttgagtatgtctcaaaatcctttatgttgaacatttctggtaatggtggtctgttcaacactctcagagagtttgtttttagaaatgtcgatgggcatttcaagatttactcaaaatttacaccagtaaacttaaatcgtggcttacctactggtctttcagtacttcaaccgttggttgaattaccagttagcataaatattactaaatttagaacactcctcactattcatagaggagaccctatgtctaataatggctggactgctttttcagctgcttatttcgtgggctatcttaaaccacgtacctttatgctgaaatataatgagaatggcaccattactgatgctgttgattgtgcacttgaccctctttcggagacaaagtgtacgttaaaatctcttagtgttcaaaagggtatctatcagacttctaactttcgagtgcaacccactcagtctatagttagatttcctaatattaccaatgtgtgtccatttcacaaggtttttaatgccacaaggtttccttctgtttacgcgtgggaaagaactaaaatttctgattgcattgcagactacactgttttctacaattcaacttctttttctactttcaaatgttatggtgtttcaccctctaaattgattgatttgtgttttacgagtgtgtatgctgatacatttctcataagattttcagaggtcagacaagtggcaccaggacagactggtgttattgctgactacaattataaattacctgatgattttacaggttgtgtcatagcctggaacacagcaaaacaggacacaggtcattatttctataggtctcatcgctctaccaaattaaaaccatttgaaagagacctttcttcagatgagaatggtgtccgtacacttagtacttatgactttaaccctaatgtaccgcttgaatatcaagctacaagggttgttgtcttgtcatttgagcttctaaatgcaccagctacagtttgtggaccaaaattatccacacaactagtaaaaaatcagtgcgttaatttcaattttaacggactcaagggcactggtgtcttgactgattcttctaagaggtttcagtcattccaacaatttggtaaagatgcgtctgactttattgattcagtacgtgatcctcaaacacttgagatacttgacattacaccttgctcttttggtggtgtcagtgttataacaccaggaacaaacacttcttcagaggtggctgttctttaccaagatgttaactgcactgatgtaccaactactatacatgcagaccaattaacacctgcttggcgcatttatgctattggcactagtgtgtttcaaactcaagcaggctgtcttataggagctgaacatgtcaatgcttcttatgagtgtgacatcccaattggtgctggtatttgtgctagctaccatacggcttctatattacgtagtacaggccagaaagctattgtggcttatactatgtcccttggtgctgagaactctattgcttatgctaacaattctatagccatacctacaaatttttcaatcagtgtcaccactgaagttatgcctgtatcaatggctaaaacttctgtagattgcactatgtatatctgcggtgactctatagagtgtagcaacttgttgttacaatatggcagtttttgcacacaactaaatcgtgctttaagtggaattgctattgaacaagacaagaacactcaagaggtttttgctcaagtcaagcaaatctataaaacaccacctattaaggattttggtggtttcaatttttcacagatattacccgatccttctaaacccagcaagaggtcgtttattgaagatttactcttcaataaagtcactcttgctgatgctggttttataaaacagtacggtgattgtttgggtgatatttctgctagagatttgatttgtgctcaaaagttcaatggactcactgtcttaccaccattgctcacagatgaaatgatcgctgcttatacagctgcattaattagcggcactgccactgctggatggacctttggtgctggtgctgctcttcaaataccatttgccatgcaaatggcttatagatttaatggaattggagttactcagaatgttctctatgagaatcagaaattaatagccaatcagtttaatagtgctattggaaaaatccaagagtctttgacatctacagctagtgcacttggaaaattgcaggatgttgttaaccaaaatgcacaagctttaaacacgcttgttaaacaacttagttccaattttggtgcaatttcaagcgtgttgaatgatattctttcacgccttgacaaagtcgaggctgaggttcagattgataggttgatcacaggtagacttcagagtttacagacgtatgtgactcaacaattaatcagagctgcagaaatcagagcttctgctaatcttgctgcgactaaaatgtccgagtgtgtactaggacaatctaaaagagttgatttttgtggaaaaggttatcacctaatgtcttttccccagtcagcgcctcatggtgttgttttcttacatgtgacttacattccttcgcaagaaaagaacttcacaacagctcctgccatttgtcatgaaggtaaagcacacttcccacgtgaaggtgttttcgtttcgaatggcacacactggtttgtaacacaaaggaacttttacgaaccccaaattataaccactgacaatacatttgtctccggtaactgtgatgttgtaattggaattatcaataacacagtttatgatcctttacaaccagaacttgattcatttaaggaggagttagataaatattttaaaaatcatacatcacctgatattgatcttggtgatatttctggcattaatgcttctgttgtcaatattcaaaaggaaattgaccgcctcaatgaggttgccagaaatttaaatgaatcactcattgatctccaagaacttggaaaatatgagcactatatcaaatggccatggtatgtttggctcggcttcattgctggactcattgctatagtcatggttacaatcctgctttgttgcatgacaagttgttgcagttgtctcaagggctgttgttcttgcggattttgctgtaaatttgatgaagatgactctgagcctgtgctcaaaggagtcaaattacattacacgtaaacgaacttatggatttgtttatgagaattttcacacttggaactgtaagtctgaaacaaggtgaaattaaggatgctactccttcagattctattcgcgctactgcaacaataccgatacaagccacactccctttcggatggcttgttgttggcgttgcaattcttgctgtttttcaaagcgcttcaaaaataattacactcaaaaagagatggcagttagccctctctaaaggtgtccactttgtttgcaacttgcttctgctgtttttaacagtttactcacacctattgcttgttgctggtggcttagaagccacttttctttttctttatgcattagcttattgcttgcaaactgtaaattttgtgagaataataatgcgattctggttgtgctggaagtgccgctccaagaatcctgtactttatgatgccaactactttctttgttggcatactaattgttatgactattgtataccatacaatagtgtaacctcttcaatcgtcatcacatgtggtgatggtactacgaatcccatttctgaggatgactaccaaattggtggttacacggaaaagtgggagtctggtgttaaggactgtgttgtattacatagttatttcacctcagactactaccagctgtactcaacacaattgagcacagacactggtgttgaacatgttactttcttcatctacaataaaattgttgatgagcctgaagaacatgtccaaattcacacaatcgacggtacatctggagttgttaatccagcaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgcttttgtggtattcttgctagtcacactagccatccttactgcgcttcgattgtgtgcgtactgctgcaatattgttaacgtgagtcttgtaaaaccttctttttacgtttactctcgtgttaaaaatctgaattcttctagagttcctgatcttttggtctaaacgaactaaatattatattagtctttctgtttggaactttaattttagccatgtcaggtgacaacggtaccattaccgttgaagagcttaaaaagctcttagaacaatggaacctagtaataggattcttgtttcttacatggatttgtttgttacaatttgcctatgccaacaggaataggtttttgtacataattaagttaattttcctctggctgctttggccagtaactttagcttgctttgtgcttgctgctgtttacagaataaactggatcactggtggaattgccattgcaatggcctgtcttgtaggcttgatgtggcttagctacttcattgcttctttcaggctgtttgctcgtacgcgttccatgtggtcatttaacccagaaactaacattcttttgaacgtgcctcttcatggcacaattctgaccaggccgcttctagagagtgaactcgtaattggagctgtgatccttcgtggacatcttcgtattgcaggacaccatctgggacgctgtgacatcaaggacctgcccaaagaaatcactgtagctacatcacgaacgctttcttattacaaattgggagcttcgcagcgtgtagcaggtgactcaggttttgctgcatacagtcgctacaggattggtaattacaaattaaatacagaccattccagtagcagtgacaatattgctttgcttgtacagtaagtgacaacagatgtttcatctcgttgactttcaggttactatagcagagatattattaattattatgaggacttttaaagtttccatttggaatcttgattatatcataaatctcataattaaaaatctatctaagcctccaactgagaataactgttctcaattagatgaagagcaaccaatggagattgattaaacgaacatgaaaattattttcttcttggtactgataacacttgttactggcgagctttaccactaccaagagtgtataaaaggtacaactgtacttttaaaagaaccttgctcttcaggaacatatgaaggcaattcaccatttcatcctctagctgataataaatttgcactgacttgctttagcactcaatttgcttttgcttgtcctgacggtgttagacacacctttcagttacgtgcgagatcagtttcacccaaactgtttaccagacaagaggaagttcaagaattatactcacctgttttccttatcgttgcagctatagtgttcataatactttgcttcacattcaaaagaaaaatagaatgagtgaattttcattaattgacttctatttgtgcttcttagcctttctgctattccttgttttaattatgctcattatcttttggttctcactagaactgcaagatcataatgaaacttctcacgcctaaacgaatatgaaatttcttgttttcttaggaattcttacaacagtagctgcattccatcaggaatgtagtttacagtcatgtgctcaacatcaaccctatgtagttgatgacccttgtccaattcacttctactcacgatggtatatcagagtgggagctagaaaatcagcacctttgattgaattgtgtgttgacgaggtaggctctaagtcacccattcaatacattgacattggtaattacacagtttcctgttctccttttacaattaattgccaggaacctaaattaggtagtctcgtagtacggtgttcgtattatgaagactttctagagtaccatgacattcgtgttgtcttagatttcatctaaacgaactaactaaaatgtctgataatggaccccaaaaccagagtagtgcaccccgcattacatttggtggaccctcagattcaagtgacaatagccaaaacggagagcgcaatggtgcacgacctaaacaacgtcgaccccaaggcttacccaataatactgcatcttggttcaccgctctcactcaacatggcaaggaaaaccttacgttccctcgagggcaaggtgttccaattaacaccaatagctctaaagatgaccaaattggctactaccgtagagctaccagacgaattcgtggcggtgacggtaaaatgaaagagctcagccccagatggtatttttactatctgggaactggaccagaagctggacttccctatggtgctaacaaagaaggcatcatatgggttgcaactgagggagccttaaacacaccaaaagaccacattggcacccgcaatcctgctaacaatgctgcaatcgtgctacaacttcctcaaggaacaacattgccaaaaggcttctacgcagaagggagcagaggcggcagtcaagcttcttcacgctcctcatcacgtagtcgcaacagttcaagaaactcaactccaggcagcagtaggggaacttctcctgctagaatggctggcaatggcggtgacactgctcttgctttgctgctgctagataggttgaaccagcttgagaacaagatatctggcaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagaattaatcagacaaggaactgattacaaacattggccgcaaattgcacaatttgctccaagtgcctctgcattctttggaatgtctcgcattggcatggaagtcacaccttcgggaacatggctgacttatcatggagccattaaattggatgacaaagatccacaattcaaagacaacgtcatactgctgaataagcacattgacgcatacaaaacattcccaccaacagagcctaaaaaggacaaaaagaaaaaggctgatgaacttcaggctttaccgcagagacagaagaagcaacaaactgtgactcttcttcctgctgcagatttggatgaattctccaaacagttgcaacaatccatgagtggtactgattcaacccaggcttaaactcatgcagaccacacaaggcagatgggctatataaacgttttcgctttccgtttacgatatatagtctactcttgtgcagaatgaattctcgtaactacatagcacaagtagatgtagttaactttaatctcacatagcaatctttaatcaatgtgtaacattagggaggatttgaaagagccaccacgttctcaccgaggccacgcggagtacgatcgagggtacagtgaataatgttagggagagcagcctatatggaagagccctaatgtgtaaaattaattttagtagtgctatccccatgtgattttaatagcttcaaccactcgacaagaaaaaaaaaaaaaaaaaaaaaaaaaa